(19) United States
(12) Patent Application Publication
(10) Pub. No.: US 2002/0010320 A1
(43) Pub. Date: Jan. 24, 2002

(54) CHEMERIC AND HUMANIZED ANTIBODIES TO ANGIOGENIN

(76) Inventor: JAMES W. FETT, WALTHAM, MA (US)

Correspondence Address: JOHN P IWANICKI, BANNER & WITCOFF LTD, 28TH FLOOR, 28 STATE STREET, BOSTON, MA, 02109

(21) Appl. No.: 09/286,240

(22) Filed: Apr. 5, 1999

(51) Int. Cl. C12P 21/08

(52) U.S. Cl. 530/387.3

(57) ABSTRACT

This invention relates to the production of chimeric and humanized antibodies that are immunologically reactive to angiogenin or to fragments thereof. This invention also relates to methods of inhibiting angiogenesis in mammals by administering chimeric and humanized antibodies, or Fab or F(ab′)₂ fragments thereof, so as to inhibit angiogenic activity. In addition, this invention relates to a pharmaceutical composition comprising therapeutically effective amounts of a chimeric or humanized antibody that is immunologically reactive with angiogenin and which can be administered to inhibit angiogenesis.

This application was funded in part by National Institutes of Health/National Cancer Institute grant no. ROl CA60046 and the Department of the Army grant no. DAMD17-96-1-6025.

### BACKGROUND OF THE INVENTION

1. Field of the Invention

Embodiments of the present invention relate in general to the production of chimeric and humanized antibodies to angiogenin. Embodiments of the present invention also relate to methods of treating tumors in humans by inhibiting angiogenesis through administration of the antibodies of the present invention in an amount effective to inhibit angiogenesis. Embodiments of the present invention further relate to methods of treating tumors in humans by inhibiting, prohibiting, reducing or eliminating tumor cell growth or otherwise inhibiting the ability of a circulating tumor cell to form a vascularized tumor mass.

2. Description of Related Art

Angiogenin is a potent inducer of angiogenesis (Fett, J. W., Strydom, D. J., Lobb, R. R., Alderman, E. M., Bethune, J. L., Riordan, J. F., and Vallee, B. L. (1985) Biochemistry 24, 5480-5486), a complex process of blood vessel formation that consists of several separate but interconnected steps at the cellular and biochemical level: (i) activation of endothelial cells by the action of an angiogenic stimulus, (ii) adhesion and invasion of activated endothelial cells into the surrounding tissues and migration toward the source of the angiogenic stimulus, and (ii) proliferation and differentiation of endothelial cells to form a new microvasculature (Folkman, J., and Shing, Y. (1992) J. Biol. Chem. 267, 10931-10934; Moscatelli, D., and Rifkin, D. B. (1988) Biochim. Biophys. Acta 948,67-85). Angiogenin has been demonstrated to induce most of the individual events in the process of angiogenesis including binding to endothelial cells (Badet, J., Soncin, F. Guitton, J. D., Lamare, O., Cartwright, T., and Barritault, D. (1989) Proc. Natl. Acad. Sci. U.S.A. 86, 8427-8431), stimulating second messengers (Bicknell, R., and Vallee, B. L. (1988) Proc. Natl. Acad. Sci. U.S.A. 85, 5961-5965), mediating cell adhesion (Soncin, F. (1992) Proc. Natl. Acad. Sci. U.S.A. 89, 2232-2236), activating cell-associated proteases (Hu, G-F., and Riordan, J. F. (1993) Biochem. Biophys. Res. Commun. 197, 682-687), inducing cell invasion (Hu, G-F., Riordan, J. F., and Vallee, B. L. (1994) Proc. Natl. Acad. Sci. U.S.A. 91, 12096-12100), inducing proliferation of endothelial cells (Hu, G-F., Riordan, J. F., and Vallee, B. L. (1997) Proc. Natl. Acad. Sci. U.S.A. 94, 2204-2209) and organizing the formation of tubular structures from the cultured endothelial cells (Jimi, S-I., Ito, K-I, Kohno, K., Ono, M., Kuwano, M., Itagaki, Y., and Isikawa, H. (1995) Biochem. Biophys. Res. Commun. 211, 476-483). Angiogenin has also been shown to undergo nuclear translocation in endothelial cells via receptor-mediated endocytosis (Moroianu, J., and Riordan, J. F. (1994) Proc. Natl. Acad. Sci. U.S.A. 91, 1677-1681) and nuclear localization sequence-assisted nuclear import (Moroianu, J., and Riordan, J. F. (1994) Biochem. Biophys. Res. Commun. 203, 1765-1772).

Although originally isolated from medium conditioned by human colon cancer cells (Fett et al., 1985, supra) and subsequently shown to be produced by several other histologic types of human tumors (Rybak, S. M., Fett, J. W., Yao, Q-Z., and Vallee, B. L. (1987) Biochem. Biophys. Res. Commun. 146, 1240-1248; Olson, K. A., Fett, J. W., French, T. C., Key, M. E., and Vallee, B. L. (1995) Proc. Natl. Acad. Sci. U.S.A. 92, 442-446), angiogenin also is a constituent of human plasma and normally circulates at a concentration of 250 to 360 ng/ml (Shimoyama, S., Gansauge, F., Gansauge, S., Negri, G., Oohara, T., and Beger, H. G. (1996) Cancer Res. 56, 2703-2706; Bläser, J., Triebl, S., Kopp, C., and Tschesche, H. (1993) Eur. J. Clin. Chem. Clin. Biochem. 31, 513-516).

While angiogenesis is a tightly controlled process under usual physiological conditions, abnormal angiogenesis can have devastating consequences as in pathological conditions such as arthritis, diabetic retinopathy and tumor growth. It is now well-established that the growth of virtually all solid tumors is angiogenesis dependent (Folkman, J. (1989) J. Natl. Cancer Inst. 82, 4-6). Angiogenesis is also a prerequisite for the development of metastasis since it provides the means whereby tumor cells disseminate from the original primary tumor and establish at distant sites (Mahadevan, V., and Hart, I. R. (1990) Rev. Oncol. 3, 97-103; Blood C. H., and Zetter B. R. (1990) Biochim. Biophys. Acta 1032, 89-118). Therefore, interference with the process of tumor-induced angiogenesis should be an effective therapy for both primary and metastatic cancers.

Several inhibitors of the functions of angiogenin have been developed. These include: (i) monoclonal antibodies (mAbs) (Fett, J. W., Olson, K. A., and Rybak, S. M. (1994) Biochemistry 33, 5421-5427) and U.S. Pat. No. 5,520,914, (ii) an angiogenin-binding protein (Hu, G-F, Chang, S-I, Riordan J. F., and Vallee, B. L. (1991) Proc. Natl. Acad. Sci. U.S.A. 88, 2227-2231; Hu, G-F., Strydom, D. J., Fett, J. W., Riordan, J. F., and Vallee B. L. (1993) Proc. Natl. Acad. Sci. U.S.A. 90, 1217-1221; Moroianu, J., Fett, J. W., Riordan, J. F., and Vallee B. L. (1993) Proc. Natl. Acad. Sci. U.S.A. 90, 3815-3819), (iii) the placental ribonuclease inhibitor (PRI) (Shapiro, R., and Vallee, B. L. (1987) Proc. Natl. Acad. Sci. U.S.A. 84, 2238-2241), (iv) peptides synthesized based on the C-terminal sequence of angiogenin (Rybak, S. M., Auld, D. S., St. Clair, D. K., Yao, Q-Z., and Fett, J. W. (1989) Biochem. Biophys. Res. Commun. 162, 535-543), and (v) inhibitory site-directed mutants of angiogenin (Shapiro, R., and Vallee, B. L. (1989) Biochemistry 28, 7401-7408). All inhibit angiogenin's activities but are not directly cytotoxic to human tumor cells grown in tissue culture.

Monoclonal antibodies (mAbs) to angiogenin or the angiogenin-binding protein when administered locally into xenografts of human tumor cells that were injected subcutaneously (s.c.) into athymic mice are able to delay or, remarkedly, completely prevent the appearance of colon, lung and fibrosarcoma tumors in these animals (Olson et al., 1995, supra, Olson, K. A., French, T. C., Vallee, B. L., and Fett, J. W. (1994) Cancer Res. 54, 4576-4579). fistological examination revealed that the mechanism of tumor growth inhibition was via an anti-angiogenesis mechanism (Olson et al., 1995, supra). Thus, the inactivation of tumor-produced angiogenin or inhibition of expression of the angiogenin gene by tumor cells promise to be a powerful means of managing cancer, either alone or in combination with more conventional therapies (i.e., chemotherapy, radiotherapy, immunotherapy, etc.).

As used herein, the term “immunoglobulin” refers to a protein consisting of one or more polypeptides substantially encoded by immunoglobulin genes. One form of immunoglobulin constitutes the basic structural unit of an antibody. This form is a tetramer and consists of two identical pairs of immunoglobulin chains, each pair having one light chain and one heavy chain. Each heavy chain has at one end a variable domain followed by a number of constant domains. Each light chain has a variable domain at one end and a constant domain at its other end, the variable domain of the light chain being aligned with the variable domain of the heavy chain and the constant domain of the light chain being aligned with the first constant domain of the heavy chain (CHI).

In each pair, the light and heavy chain variable regions are together responsible for binding to an antigen, and the constant regions are responsible for the antibody effector functions. In addition to antibodies, immunoglobulins may exist in a variety of other forms including, for example, Fv (fragment-variable), Fab (fragment-antigen binding) or F(ab′)s which represents two Fab′ arms linked together by disulfide bonds. The light chain constant domain and the CH1 domain of the heavy chain account for 50% of each Fab′ fragment. The “tail” or central axis of the antibody contains a fixed or constant sequence of peptides and is termed the Fc fragment (fragment-crystalline). Other antibodies include bifunctional hybrid antibodies (e.g., Lanzavecchia et al., Eur J. Immunol. 17,105 (1987)) and those in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology,” Benjamin, N.Y., 2nd ed. (1984), and Hunkapiller and Hood, Nature, 323-15-16 (1986), which are incorporated herein by reference).

The variable domains of each pair of light and heavy chains form the antigen binding site. The variable domains on the light and heavy chains have the same general structure and each domain comprises four framework regions (FRs), whose sequences are relatively conserved, connected by three complementarity determining regions (CDRs). See Kabat et al., Sequences of Proteins of Immunological Interest, U.S. Department of Health and Human Services (1987). The four framework regions largely adopt a beta-sheet conformation and the CDRs form loops connecting, and in some cases forming part of, the beta-sheet structure. The CDRs are held in close proximity by the framework regions and, with the CDRs from the other domain, contribute to the formation of the antigen binding site. As used herein, a “human framework region” is a framework region that is substantially identical (about 85% or more, usually 90-95% or more) to the framework region of a naturally occurring human immunoglobulin.

The recognized immunoglobulin genes include the kappa, lambda, alpha, gamma (IgG₁, IgG₂, IgG₃, IgG₄), delta, epsilon and mu constant region genes, as well as the myriad of immunoglobulin variable region genes. Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 10 or more amino acids, with the heavy chain also including a “D” region of about 12 more amino acids. (See, generally, Fundamental Immunology, Paul, W., Ed., Chapter 7, pages. 131-166, Raven Press, N.Y. (1984), which is incorporated herein by reference). Full-length immunoglobulin light chains (about 25 Kd or 214 amino acids), are encoded by a variable region gene at the NH2-terminus (about 110 amino acids) and a kappa or lambda constant region gene at the COOH-terminus. Full-length immunoglobulin heavy chains (about 50-70 Kd or 446 amino acids), are similarly encoded by a variable region gene (about 116 amino acids) and one of the other aforementioned constant region genes, e.g., gamma (encoding about 330 amino acids). The NH₂-terninus of each chain begins a variable region of about 100 or 110 or more amino acids primarily responsible for antigen recognition. The COOH terminus of each chain defines a constant region primarily responsible for effector function.

The production of monoclonal antibodies was first disclosed by Kohler and Milstein (Kohler & Milstein, Nature, 256, 495-497 (1975)). Monoclonal antibodies have found widespread use as diagnostic and therapeutic agents. Human hybridomas which secrete human antibody can be produced by the methods disclosed in Kohler and Milstein, supra. Although human antibodies are especially preferred for the treatment of humans in general, creation of stable human-human hybridomas for long term production of human monoclonal antibody can be difficult. However, hybridoma production in rodents, especially mouse, is an established procedure. Stable murine hybridomas provide an unlimited source of antibody of select characteristics. Murine antibodies, however, may have limited use in the treatment of humans as they can be highly immunogenic.

As an alternative, chimeric antibodies may be prepared having a variable or antigen binding (hypervariable or complementarity determining) region derived from an animal (e.g. a mouse) antibody and the remaining regions derived from a human antibody. Methods for producing chimeric (e.g., murine/human) antibodies are described according to the teachings of the present invention below. Chimeric antibodies can be produced in large quantities and they are less immunogenic in humans than non-human antibodies. They are better suited for in vivo administration than animal antibodies, especially when repeated or long term administration is necessary.

As a further alternative, humanized antibodies may be prepared having an antigen binding (hypervariable or complementarity determining) region derived from an animal (e.g. a mouse) antibody and the remaining framework and constant regions derived from a human antibody. The non-human immunoglobulin providing the CDRs is called the “donor” and the human immunoglobulin providing the framework is called the “acceptor.” Constant regions need not be present, but if they are, they are generally substantially identical to human immunoglobulin constant regions, i.e. at least about 85-90% identical. Hence, all parts of a humanized immunoglobulin, except possibly the CDRs, are substantially identical to corresponding parts of natural human immunoglobulin sequences. A “humanized antibody” is therefore an antibody comprising a humanized variable region on the light chain and the heavy chain immunoglobulin. For example, a humanized antibody is distinguishable from a chimeric antibody as defined above, e.g., because the entire variable region of a chimeric antibody is non-human.

Recombinant DNA technology has been utilized to produce immunoglobulins which have human framework regions combined with complementarity determining regions from a donor mouse or rat immunoglobulin (see, EPO publication no. 0239400, which is incorporated herein by reference in its entirety). Methods for producing humanized antibodies are described according to the teachings of the present invention below. Humanized antibodies can be produced in large quantities and they are even still less immunogenic in humans than chimeric or non-human antibodies. They are even better suited for in vivo administration than chimeric or animal antibodies, especially when repeated or long term administration is necessary.

Accordingly, a need exists to develop more efficacious and potent inhibitors of angiogenin as a method of treating diseases associated with abnormal angiogenesis, such as tumor growth. A need further exists to develop partially or wholly humanized antibodies to angiogenin having a strong affinity to angiogenin and which are useful in inhibiting angiogenesis. These partially or wholly humanized antibodies to angiogenin should remain substantially non-immunogenic in humans, yet be easily and economically produced in a manner suitable for therapeutic formulation and other uses.

### SUMMARY OF THE INVENTION

Embodiments of the present invention relate to the production of novel antibodies which have been partly or wholly humanized and are immunologically reactive to angiogenin. The antibodies of the present invention have generally one or more variable regions from a nonhuman donor immunoglobulin in combination with human constant regions. In an alternate embodiment of the present invention, the antibodies have generally one or more complementarity determining regions (CDRs) from a nonhuman donor immunoglobulin and a framework region from a human immunoglobulin. The antibodies include an antigen binding site of non-human source which retains its antigen binding affinity for angiogenin although being combined with constant and/or framework regions of human source. In particular, embodiments of the present invention include characterizing the hypervariable regions of the antigen binding site of an antibody immunologically reactive with angiogenin and providing these CDRs within an antibody with portions thereof having human origin.

The antibodies of the present invention have important therapeutic and diagnostic importance in the treatment of conditions associated with abnormal angiogenesis.

Further features and advantages of the invention will become more fully apparent in the following description of the embodiments and drawings thereof, and from the claims.

### BRIEF DESCRIPTION OF DRAWINGS

FIG. 1 is a nucleotide and corresponding amino acid sequence for the light chain variable region and heavy chain variable region for the murine monoclonal antibody 26-2F (mAb 26-2F). The sequences were determined according to Kabat, E. A., Wu, T. T., Perry, H. M., Gottesman, K. S. & Foeller, C. (1991) U.S. Department of Health and Human Services, NIH publication no. 91-3242, 5th ed. hereby incorporated by reference in its entirety.

FIG. 2 depicts the nucleic acid sequence of the entire human angiogenin gene including the cDNA sequence as identified by arrows along with the deduced amino acid sequence.

FIG. 3 is a photograph of a Western blot analysis of the chimeric antibody of the present invention, cAb 26-2F versus molecular weight standards (x 10⁻³). Lane 1 represents mAb 26-2F, lane 2 represents cAb 26-2F from cell line S 13-1 and lane 3 represents cAb 26-2F from cell line P4-5. Heading A represents incubation of proteins with goat anti-human K chain antibodies. Heading B represents incubation of proteins with goat anti-human IgG Fc-specific antibodies followed by treatment with alkaline phosphatase-labeled rabbit anti-goat IgG and nitroblue tetrazolium.

FIG. 4 is a graph of the inhibition of ribonucleolytic activity of angiogenin by mAb 26-2F, cAb 26-2F and a control MOPC 31C.

FIG. 5 is a graph showing prevention of MDA-MB-435 (A) and MCF-7 (B) tumor formation by mAb 26-2F or cAb 26-2F.

### DETAILED DESCRIPTION OF CERTAIN PREFERRED EMBODIMENTS

The principles of the present invention may be advantageously applied to produce novel antibodies to angiogenin which are immunologically reactive with angiogenin. The novel antibodies are partly or wholly humanized and are directed in particular to the angiogenin protein such that they inhibit angiogenesis. The novel antibodies of the present invention have important therapeutic and diagnostic implications.

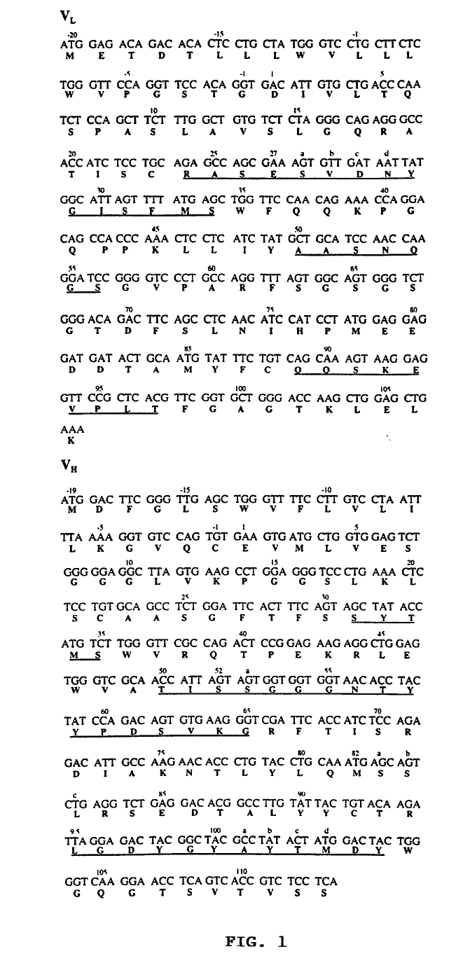

According to one embodiment of the present invention, a partly humanized, i.e. chimeric, or wholly humanized antibody immunologically reactive to angiogenin is produced by expressing recombinant DNA segments encoding the heavy and light chain variable regions, or more particularly CDRs, from a donor immunoglobulin capable of binding to angiogenin, attached to DNA segments encoding the heavy and light chain constant regions and/or framework regions from a human source. Due to codon degeneracy and non-critical amino acid substitutions, other DNA sequences can be readily substituted for those sequences, as described in FIG. 1.

The DNA segments will typically further include an expression control DNA sequence operably linked to the chimerized or humanized immunoglobulin coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host, the host is maintained under conditions suitable for high level expression of the nucleotide sequences, and, as desired, the collection and purification of the chimeric or humanized light chains, heavy chains, light/heavy chain dimers or intact antibodies, binding fragments or other immunoglobulin forms may then follow (see, S. Beychok, Cells of Immunoglobulin Synthesis, Academic Press, N.Y., (1979), which is incorporated herein by reference).

Human constant region DNA sequences can be isolated in accordance with well known procedures from a variety of human cells, but preferably immortalized B-cells (see, Kabat et al. and WO 87/02671). The variable regions or CDRs for producing the immunoglobulins of the present invention will be similarly derived from monoclonal antibodies capable of binding to angiogenin and produced by well known methods in any convenient mammalian source including, mice, rats, rabbits, or other vertebrates, capable of producing antibodies. Suitable source cells for the constant region and framework DNA sequences, and host cells for immunoglobulin expression and secretion, can be obtained from a number of sources, such as the American Type Culture Collection (“Catalogue of Cell Lines and Hybridomas,” sixth edition (1988) Rockville, Md., U.S.A., which is incorporated herein by reference).

In addition to the chimerized or humanized immunoglobulins to angiogenin specifically described herein, other “substantially homologous” modified immunoglobulins to the native sequences can be readily designed and manufactured utilizing various recombinant DNA techniques well known to those skilled in the art. For example, the variable or framework regions can vary specifically from the sequences described herein at the primary structure level by several amino acid substitutions, terminal and intermediate additions and deletions, and the like. Moreover, a variety of different human framework regions may be used singly or in combination as a basis for the chimerized or humanized immunoglobulins of the present invention. Also, the variable or CDR regions may be antigenic to proteins similar in structure to angiogenin and also inhibit the angiogenic activity of angiogenin. In general, modifications of the genes may be readily accomplished by a variety of well-known techniques, such as site-directed mutagenesis (see Gillman and Smith, Gene, 8, 81-97 (1979) and S. Roberts et al., Nature, 328, 731-734 (1987), both of which are incorporated herein by reference). In addition, the amino acid sequences described herein may be modified by the substitution or deletion of one or more amino acids which do not substantially effect the ability of the antibody to bind to angiogenin, such as by conservative substitution and still remain substantially homologous.

Substantially homologous immunoglobulin sequences are those which exhibit at least about 85% homology, usually at least about 90%, and preferably at least about 95% homology with a reference immunoglobulin protein, such as the sequences specifically identified herein.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in appropriate vectors using site-directed mutagenesis, such as after CHI (the first constant region on the heavy chain) to produce Fab fragments or after the hinge region to produce (Fab′)₂ fragments. Single chain antibodies may be produced by joining the light chain variable region and the heavy chain variable region with a DNA linker.

The nucleic acid sequences of the present invention capable of ultimately expressing the desired chimerized or humanized antibodies can be formed from a variety of different polynucleotides (genomic or cDNA, RNA, synthetic oligonucleotides, etc.) and components (e.g., V, J, D, and C regions), as well as by a variety of different techniques. Joining appropriate synthetic and genomic sequences is presently the most common method of production, but cDNA sequences may also be utilized (see, European Patent Publication No. 0239400 and L. Reichmann et al., Nature, 332, 323-327 (1988), both of which are incorporated herein by reference).

As stated previously, the DNA sequences are expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors contain selection markers, e.g., tetracycline or neomycin, to permit detection of those cells transformed with the desired DNA sequences (see e.g., U.S. Pat. No. 4,704,362, which is incorporated herein by reference).

E. coli is one prokaryotic host useful particularly for cloning the DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any number of a variety of well-known promoters will be present, such as the lactose promoter system, a tryptophan (trp) promoter system, a β-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, mammalian tissue cell culture may also be used to express and produce the polypeptides of the present invention (see, Winnacker, “From Genes to Clones,” VCH Publishers, N.Y., N.Y. (1987), which is incorporated herein by reference). Eukaryotic cells are actually preferred, because a number of suitable host cell lines capable of secreting intact immunoglobulins have been developed in the art, and include the CHO cell lines, various COS cell lines, HeLa cells, preferably myceloma cell lines, etc, and transformed B-cells or hybridomas. Expression vectors for these cells can include expression control sequences, such as an origin of replication, a promoter, an enhancer (Queen et al., Immunol. Rev., 89 49-68 (1986), which is incorporated herein by reference), and necessary processing information sites, such as ribosome binding sites, RNA splice sites, polyadenylation sites, and transcriptional terminator sequences. Preferred expression control sequences are promoters derived from immunoglobulin genes, SV40, adenovirus, cytomegalovirus, bovine papilloma virus, and the like.

The vectors containing the DNA segments of interest (e.g., the heavy and light chain encoding sequences and expression control sequences) can be transferred into the host cell by well-known methods which vary depending on the type of cellular host. For example, calcium chloride transfection is commonly utilized for prokaryotic cells, whereas calcium phosphate treatment or electroporation may be used for other cellular hosts. (See, generally, Maniatis et al., Molecular Cloning: A Laboratory Manual, Cold Spring Harbor Press, (1982), which is incorporated herein by reference).

Once expressed, the whole antibodies, their dimers, individual light and heavy chains, or other immunoglobulin forms of the present invention, can be purified according to standard procedures of the art, including ammonium sulfate precipitation, affinity columns, column chromatography, gel electrophoresis and the like (see, generally, R. Scopes, “Protein Purification,” Springer-Verlag, N.Y. (1982)). Substantially pure immunoglobulins of at least about 90 to 95% homogeneity are preferred, and 98 to 99% or more homogeneity most preferred, for pharmaceutical uses. Once purified as desired, the polypeptides may then be used therapeutically (including extracorporeally) or in developing and performing assay procedures, immunoflourescent stainings, and the like. (See, generally, Immunological Methods, Vols. I and II. Lefkovits and Pernis, eds., Academic Press, New York, N.Y. (1979 and 1981)).

The present invention also provides novel compositions useful, for example, in the treatment of conditions associated with abnormal angiogenesis. The compositions include the chimeric or humanized immunoglobulins immunologically reactive with angiogenin. The immunoglobulins can have two pairs of light chain/heavy chain complexes, typically at least one chain including a mouse variable region or at least mouse complementarity determining regions functionally joined to human framework region segments. For example, mouse complementarity determining regions, with or without additional naturally-associated mouse amino acid residues, can be used to produce human-like antibodies capable of binding to angiogenin at affinity levels stronger than about 10⁸ M⁻¹, and preferably 10⁹ M⁻¹ to 10¹⁰ M⁻¹ or stronger, and capable of inhibiting the angiogenic activity of angiogenin.

The immunoglobulins, including binding fragments and other derivatives thereof, of the present invention may be produced readily by a variety of recombinant DNA techniques, with ultimate expression in transfected cells, preferably immortalized eukaryotic cells, such as myeloma or hybridoma cells. Polynucleotides comprising a first sequence coding for non-human immunoglobulin variable or complementarity determining regions and a second sequence set coding for the desired human immunoglobulin framework or constant regions can be produced synthetically or by combining appropriate cDNA and genomic DNA segments.

The chimeric or humanized immunoglobulins of the present invention may be utilized alone in substantially pure form, or complexed with other therapeutically active agents or cytotoxic agents, such as a radionuclide, a ribosomal inhibiting protein or a cytotoxic agent active at cell surfaces. The chimeric or humanized immunoglobulins or their complexes can be prepared in a pharmaceutically accepted dosage form, which will vary depending on the mode of administration.

Angiogenesis is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. The present invention is directed towards prevention of angiogenesis in the treatment of these and other angiogenesis dependent tumors and the resultant damage to the mammal due to the presence of the tumor.

Angiogenesis is also associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in bone marrow that gives rise to leukemia-like tumors.

Angiogenesis is important in two stages of tumor metastasis. The first stage where angiogenesis stimulation is important is in the vascularization of the tumor which allows cells to enter the blood stream and to circulate throughout the body. After the tumor cells have left the primary site, and have settled into the secondary, metastasis site, angiogenesis must occur before the new tumor can grow and expand. Therefore, embodiments of the present invention are directed to the inhibition of angiogenesis as a treatment for the prevention of metastasis of tumors and containment of the neoplastic growth at the primary or secondary site.

Accordingly, the antibodies of the present invention will typically find use in methods of reducing, eliminating, inhibiting or otherwise interfering with the angiogenic activity of angiogenin. The antibodies of the present invention are immunologically reactive with angiogenin, i.e. capable of binding to angiogenin in a manner to inhibit the angiogenic activity of angiogenin. In accordance with one aspect of the present invention, the antibodies of the present invention are administered to an individual having a condition associated with abnormal angiogenesis so as to bind to the angiogenin in a manner to inhibit the angiogenic activity of angiogenin.

Embodiments of the present invention are also directed to methods for reducing size of tumors associated with angiogenesis in a mammal comprising administering to the mammal an effective amount of an antibody of the present invention so as to reduce tumor size. Embodiments of the present invention are still further directed to methods for inhibiting metastasis of tumor cells in a mammal comprising administering to the mammal an effective amount of an antibody of the present invention so as to inhibit metastasis of tumor cells. Embodiments of the present invention are even still further directed to methods for inhibiting the establishment of tumor cells in a mammal comprising administering to the mammal an effective amount of an antibody of the present invention so as to inhibit establishment of tumor cells. Embodiments of the present invention are even still further directed to methods for inhibiting growth of tumors associated with angiogenesis in a mammal comprising administering to the mammal an effective amount of an antibody of the present invention so as to inhibit tumor growth. The antibodies and methods described herein are therefore useful in methods of therapeutically treating a mammal, including a human, afflicted with pathological conditions associated with abnormal or unwanted angiogenesis, including cancer.

Examples of diseases mediated by angiogenesis are disclosed in the prior art such as U.S. Pat. No. 5,712,291 and include ocular neovascular disease as well as the other diseases to follow. Ocular neovascular disease is characterized by invasion of new blood vessels into the structure of the eye such as the retina or comea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, mycobacteria infections, lipid degeneration, chemical burns, bacterial ulcers, fungal ulcers, Herpes simples infections, Herpes zoster infections, protozoan infections, Kaposi sarcoma, Mooren ulcer, Terrien's marginal degeneration, marginal keratolysis, rheumatoid arthritis, systemic lupus, polyarteritis, trauma, Wegener's sarcoidosis, Scleritis, Steven Johnson's disease, periphigoid radical keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering from arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed with the lumen of blood vessels have been shown to have angiogenic stimulatory activity.

One of the most frequent angiogenic diseases of childhood is hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding sometimes with pulmonary or hepatic arteriovenous fistula. For example, typical disease states suitable for treatment include graft versus host disease and transplant rejection in patients undergoing an organ transplant, such as heart, lungs, kidneys, liver, etc. Other diseases include autoimmune diseases, such as Type I diabetes, multiple sclerosis, rheumatoid arthritis, systemic lupus erythematosus, and myasthenia gravis.

The chimeric or humanized antibodies of the present invention may also be used in combination with other antibodies, particularly human monoclonal antibodies reactive with other antigens present in conditions associated with abnormal angiogenesis. The chimeric and humanized antibodies and pharmaceutical compositions thereof of this invention are particularly useful for parenteral administration, i.e., subcutaneously, intramuscularly or intravenously. The compositions for parenteral administration will commonly comprise a solution of the antibody or a cocktail thereof dissolved in an acceptable carrier, preferably an aqueous carrier. A variety of aqueous carriers can be used, e.g., water, buffered water, 0.4% saline, 0.3% glycine and the like. These solutions are sterile and generally free of particulate matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, human albumin, etc. The concentration of antibody in these formulations can vary widely, i.e. from less than about 0. 5%, usually at or at least about 1% to as much as 15 or 20% by weight and will be selected primarily based on fluid volumes, viscosities, etc., in accordance with the particular mode of administration selected.

Thus, a typical pharmaceutical composition for injection could be made up to contain 1 ml sterile buffered water, and 1 to 50 mg of antibody. A typical composition for intravenous infusion could be made up to contain 250 ml of sterile Ringer's solution, and 150 mg of antibody. Actual methods for preparing parenterally administrable compositions will be known or apparent to those skilled in the art and are described in more detail in, for example, Remington's Pharmaceutical Science, 15th ed., Mack Publishing Company, Easton, Pa. (1980), which is incorporated herein by reference.

The immunotherapeutic agents of this invention are chimeric and humanized antibodies, Fab and F(ab′)₂ fragments thereof, and mixtures thereof which are immunologically reactive with angiogenin and/or with natural and/or synthetic peptide fragments of angiogenin. These immunotherapeutic agents are useful medicaments in the treatment of pathological processes in mammals where angiogenesis is an undesired manifestation of the process. Because these immunotherapeutic agents can inhibit angiogenesis, they are particularly useful in the treatment of tumors in mammals.

As pharmaceutical compositions, the immunotherapeutic agents of this invention can be administered in a wide variety of dosage forms, either alone or in combination with other pharmaceutically compatible medicaments, and in the form of pharmaceutical compositions suited for systemic or localized injection, time release implants and the like.

Typically, the immunotherapeutic agents of this invention are administered in the form of pharmaceutical compositions suited for injection consisting essentially of the free antibody and a pharmaceutical carrier.

The pharmaceutical carrier can either be a solid or semi-solid material, or a liquid in which the immunotherapeutic agent is dissolved, dispersed or suspended and which can optionally contain small amounts of pH buffering agents and/or preservatives. Suitable buffering agents include for example sodium acetate and pharmaceutical phosphate salts and the like. Pharmaceutically acceptable preservatives include for example benzyl alcohol and the like.

Representative of pharmaceutically effective dosage ranges are 6.6 μg to 66 μg of antibody/dose. However, therapeutically effective dosage ranges can be expected to vary based upon the avidity of the particular antibody selected, the size, age and weight of the patient being treated, and the like, and can readily be determined by simple experiment.

The antibodies of this invention can be frozen or lyophilized for storage and reconstituted in a suitable carrier prior to use. This technique has been shown to be effective with conventional immune globulins and art-known lyophilization and reconstitution techniques can be employed. It will be appreciated by those skilled in the art that lyophilization and reconstitution can lead to varying degrees of antibody activity loss (e.g., with conventional immune globulins, IgM antibodies tend to have greater activity loss than IgG antibodies) and that use levels may have to be adjusted to compensate.

The compositions containing the present chimeric or humanized antibodies or a cocktail thereof can be administered for prophylactic and/or therapeutic treatments. In therapeutic application, compositions are administered to a patient already suffering from a disease, in an amount sufficient to cure or at least partially arrest the condition and its complications. An amount adequate to accomplish this is defined as a “therapeutically effective dose.” Amounts effective for this use will depend upon the severity of the condition, but generally range from about 1 to about 200 mg of antibody per dose, with dosages of from 5 to 25 mg being more commonly used.

In prophylactic applications, compositions containing the present antibodies or a cocktail thereof are administered to a patient not already in a condition associated with abnormal angiogenesis. Such an amount is defined to be a “prophylactically effective dose.” In this use, the precise amounts again depend upon the patient's state of health, but generally range from 0.1 to 25 mg per dose, especially 0.5 to 2.5 mg per dose. A preferred prophylactic use is for prevention of the metastasis of existing tumor cells or the inhibition of the ability of circulating tumor cells to form a vascularized tumor mass.

Single or multiple administrations of the compositions can be carried out with dose levels and pattern being selected by the treating physician. In any event, the pharmaceutical formulations will provide a quantity of the antibody(ies) of this invention sufficient to effectively treat the patient.

The chimeric or humanized antibodies of the present invention can further find a wide variety of utilities in vitro. By way of example, the antibodies can be utilized for detecting the presence of angiogenin or fragments thereof in a sample, for vaccine preparation, or the like.

Kits can also be supplied for use with the subject antibodies in the protection against or detection of a cellular activity or for the presence of angiogenin. Thus, the subject antibody composition of the present invention may be provided, usually in a lyophilized form in a container, either alone or in conjunction with additional antibodies to specific antigens associated with angiogenesis. The antibodies, which may be conjugated to a label or toxin, or unconjugated, are included in the kits with buffers, such as Tris, phosphate, carbonate, etc., stabilizers, biocides, inert proteins, e.g., serum albumin, or the like, and a set of instructions for use. Generally, these materials will be present in less than about 5% wt based on the amount of active antibody, and usually present in total amount of at least about 0.001% wt based again on the antibody concentration. Frequently, it will be desirable to include an inert extender or excipient to dilute the active ingredients, where the excipient may be present in from about 1 to 99% wt of the total composition. Where a second antibody capable of binding to the chimeric or humanized antibody is employed in an assay, this will usually be present in a separate vial. The second antibody is typically conjugated to a label and formulated in an analogous manner with the antibody formulations described above.

The following examples are set forth as representative of the present invention. These examples are not to be construed as limiting the scope of the invention as these and other equivalent embodiments will be apparent in view of the present disclosure, figures, tables, and accompanying claims.

### EXAMPLE I

### Construction and Characterization of the Murine mAb 26-2F

A monoclonal antibody immunologically reactive with angiogenin identified as mAb 26-2F (ATCC No. HB9766) was produced according to the procedure disclosed in U.S. Pat. No. 5,520,914 hereby incorporated by reference in its entirety. Specifically, Balb/c mice were initially injected with 30 μg of angiogenin in complete Freund's adjuvant subcutaneously. Two more injections of 30 μg of angiogenin in incomplete Freund's adjuvant were given 10 days and 17 days after the initial injection. Three days before the fusion, mice were boosted with 30 μg of angiogenin in normal saline given as an intraperitoneal injection.

On the day of the fusion, the mouse boosted with the angiogenin in saline was sacrificed and the spleen harvested. A suspension of the spleen cells was obtained by purging the spleen with serum-free medium using a 22 gauge needle. The spleen cells were washed three times with 10 ml/wash of serum-free media. The Sp2/0 or P3x63-Ag 8.653 fusion partner myeloma cells to be used were also washed three times with serum-free medium. The spleen cells were mixed with the myeloma cells at a 4:1 spleen to myeloma cell ratio. The spleen-myeloma cell mixture was pelleted by centrifugation and placed in a water bath at 37° C. One ml of filtered polyethylene glycol (PEG, 0.83 mg/mil in serum-free media) was added slowly over a 30 second interval. After gentle mixing of the cells and PEG for 90 seconds, 5 ml of serum-free media was added over a 5 minute period followed by a 14 ml of HAT media over a 1 minute period. The cells were then centrifuged for 7 minutes and the supernatant discarded. The cells were suspended in HAT media containing mouse peritoneal exudate cells (4×10⁵ cells/plate) and plated into 96-well tissue culture plates at 200 μl per well. Seven days after the fusion the HAT medium was removed and replaced with HT medium. Wells were checked daily for colony growth. Supernatants from those wells exhibiting colony growth were assayed for angiogenin-binding antibodies by ELISA. Cells yielding supernatant containing angiogenin-binding antibodies were subcloned twice by limiting dilution.

Alternatively, mAb 26-2F was produced in ascites fluid in Charles River nu/nu mice according to the following procedure. Charles River nu/nu mice are primed with a 1 ml intraperitoneal injection of pristane followed 7 days later with an intraperitoneal injection of 1×10⁶ hybridoma cells. Ascites fluid is collected from the mice 1 to 2 weeks later, centrifuged to remove cells and frozen for subsequent purification. Antibodies in the filtered hybridoma-conditioned media or ascites are precipitated by saturated ammonium sulfate, centrifuged to a pellet, decanted, resuspended in saturated ammonium sulfate, pelleted again, resuspended in normal saline (0.15 M NaCl, pH 7.4) and finally dialyzed against normal saline (0.15 M NaCl, pH 7.4) The resulting solution is purified further by Protein A-Sepharose chromatography, dialyzed against normal saline, sterile filtered and stored in aliquots at −70° C.

Hybridoma-conditioned medium or ascites fluid was clarified by filtration through Whatman glass fiber filters. Equal volumes of saturated ammonium sulfate were added dropwise to the clarified hybridoma-conditioned medium and stirred for 1 hr at room temperature. The mixture was centrifuged (10,000 g) for 10 min and the resulting pellet resuspended in saturated ammonium sulfate and washed by centrifugation. Pelleted material was resuspended in normal saline and dialyzed as above against normal saline.

Clarified hybridoma-conditioned medium or ascites fluid was dialyzed overnight at 4° C. in 6000-8000 MW cutoff bags against 50 volumes 0.1 M sodium phosphate buffer, pH 8.0. This material was applied to a 5 ml bed of Protein A-Sepharose. Unbound material was washed from the column using 0.1 M sodium phosphate buffer, pH 8.0, and Ig enriched fraction eluted from the column in 0.1 M sodium citrate buffer, pH 3.5, and dialyzed against normal saline. The resultant fraction was highly purified, as evidenced by gel electrophoresis under reducing conditions which revealed as major bands only the light and heavy chains of immunoglobulins.

Alternatively, the mAb 26-2F was purified from ascites fluid by affinity chromatography using GammaBind Plus Sepharose (Pharmacia). Ascites fluid (80 ml) was diluted 1:1 with PBS, and clarified by centrifugation. The supernatant was filtered through a glass fiber filter and a 0.2 μm cellulose nitrate filter. After a further dilution with PBS to a final volume of 400 ml, the antibodies were adsorbed onto the gel, washed with PBS, and eluted with 0.1 M glycine-HCl into tubes containing an appropriate amount of 1 M Tris-HCl to neutralize the supernatant. Following dialysis against 0.9% NaCl, the antibodies were quantified by enzyme-linked immunoadsorbent assay and stored at −70° C. MOPC 3 IC, a non-specific IgGIκ-secreting mouse hybridoma (CCL 130, American Type Culture Collection) was propagated, and IgG purified from ascites as above.

### EXAMPLE II

### Characterization of mAb 26-2F

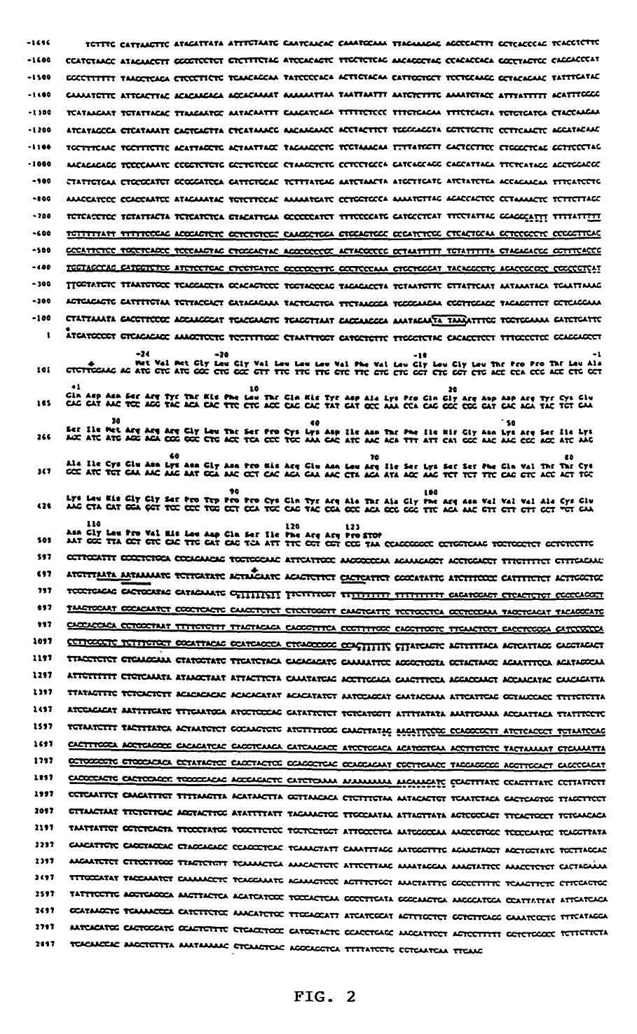

The mAb 26-2F was the result of the fusion of the spleen cells of an angiogenin-immunized Balb/c mouse with the P3x63-Ag8.653 myeloma line. Monoclonal antibodies were characterized by ELISA and radio-immunoassay (RIA) for their ability to recognize other species of angiogenin such as bovine, porcine, and rabbit-derived angiogenin. In addition, mutants of angiogenin produced in our laboratory were used to characterize further the epitope binding of the monoclonal antibodies. The entire amino acid sequence for human angiogenin is shown in FIG. 2. One set of mutants consisted of single amino acids substitutions: the lysine at residue 40 substituted with either glutamine (K40Q) or arginine (K40R), the arginine with residue 66 substituted with alanine (R66A), tryptophan at residue 89 substituted with methionine (W89M), and the aspartic acid at residue 116 substituted with histidine (D116H) or alanine (D116A). The other set of mutants employed consisted of angiogenin-bovine RNase A hybrids (ARH): ARH in which angiogenin residues 58-70 are replaced with RNase residues 59-73 (ARH-1), ARH in which angiogenin residues 38-41 are replaced with RNase residues 38-42, (ARH-2), ARH in which ARH-1 is further substituted with angiogenin residues 8-22 replaced with RNase A residues 7-21 (ARH-3) and ARH in which angiogenin residues 8-22 are replaced with RNase A residues 7-21 (ARH-4).

The mAb 26-2F is characterized as IgGl 78 with a binding affinity of 1.6 nM which recognizes a discontinuous epitope in angiogenin involving Trp-89 and residues in the segment 38-41, located in two adjacent loops of the angiogenin dimensional structure identified in Fett, J. W., Olson, K. A. & Rybak, S. M. (1994) Biochemistry 33, 5421-5427 and Acharya, K. R., Shapiro, R., Allen, S. C., Riordan, J. F. & Vallee, B. L. (1994) Proc. Nat'l. Acad Sci. USA 91, 2915-2929 each hereby incorporated by reference in their entireties. It binds strongly to angiogenin both in a solid phase ELISA and a soluble phase (RIA). The mAb 26-2F neutralizes the ribonucleolytic, angiogenic, and mitogenic activities of human angiogenin. The mAb 26-2F also interferes with the establishment and metastatic spread of tumors cells in athymic mice. It does not bind to bovine, porcine, or rabbit angiogenin in a soluble phase inhibition assay for binding to iodinated angiogenin. In this same assay, mAb 26-2F binds equally well to the D116A mutant as to native angiogenin and the binding to K40Q and K40R is only decreased by approximately two fold. In examining the interaction of the RNase-sequence containing mutants, mAb 26-2F binds equally well to the ARH-1, ARH-3 and ARH-4 mutants as to the native angiogenin. However, in the inhibition RIA, mAb 26-2F does not recognize the ARH-2 mutant.

### EXAMPLE III

### Molecular Modeling of mAb 26-2FlAngiogenin Complex

A data set to 2.8 A has been collected on the mAb 26-2F Fab fragment/angiogenin complex. Fab and angiogenin were positioned in the unit cell using the method of molecular replacement (Navaza, J. (1994) Acta Crystallogr. A50, 157-163). Refinement of the structure was carried out using protocols that resulted in decreases in both R_{cryst} and R_{free} in order to avoid overfitting. The CDRs were not included in the inital model but were slowly incorporated when sufficient density permitted. The current model has an R_{cryst} and R_{free} of 33% with good stereochemistry.

The complex is formed through contacts between the CDRs of the Fab and two flexible loops of angiogenin comprising the residues 37-41 and 85-89. The entire amino acid sequence for human angiogenin is shown in FIG. 2. In particular, side-chain terminal atoms of residues Asn-27d and Tyr-28 from the CDR1 region of the V_{L} Fab domain (L1) and Tyr-100B from the CDR3 region of the V_{H} Fab domain (H3) form strong hydrogen bonds with main-chain atoms (carbonyl oxygens) from the angiogenin 37-39 loop. Tyr-59 from the H2 domain of the Fab fragment also forms a hydrogen bond with the carbonyl oxygen of Trp-89 of angiogenin. In addition, hydrophobic interactions appear to further stabilize the Fab-angiogenin complex. These interactions are mediated through residues from the H3, L1 and L2 regions. Upon complex formation, the two angiogenin loops involved in binding move slightly in order to optimize contacts with the Fab. However, the region between the disulfide bond formed between cysteine residues 39 and 92 of angiogenin is not affected. The total accessible area buried upon complex formation is ˜1360 Å².

### EXAMPLE IV

### Isolation and Analysis of Variable Region cDNAs

To produce chimeric antibodies that are immunologically reactive with angiogenin, the variable regions from a non-human monoclonal antibody that is immunologically reactive with angiogenin must be isolated. In particular, the variable regions of mAb 26-2F were cloned, sequenced and subcloned into expression vectors according to the methods described below.

Polyadenylated RNA was prepared from mAb 26-2F producing hybridoma cells using the PoliATtract System 1000 mRNA isolation kit (Promega). The light chain variable region (V_{L}) cDNA and the heavy chain variable region (V_{H}) cDNA were isolated by a modified version of the method of Coloma et al. (1992) J. Immunol. Methods 152, 89-104 hereby incorporated by reference in its entirety.

The primers used to isolate V_{L} and V_{H} cDNAs were designed to minimize the introduction of mutations affecting chimeric antibody activity into the V_{L} or V_{H} cDNAs. The first strand of a V_{H} specific cDNA was synthesized by the method of reverse transcriptase polymerase chain reaction (RT-PCR) using the C_{H}1 antisense primer Mγ C.C_{H}1 AS as follows:

(5′AGGTCTAGAA(CT)CTCCACACACAGG(AG)(AG)CCAGTGGATAGAC)

and AMV reverse transcriptase (Promega). V_{H} cDNA amplification was performed using Mγ C.C_{H}1 AS as the antisense primer and a set of three universal sense primers that are complementary to the N-termini of most V_{H} leader sequences such as MHALT1.RV, MHALT2.RV, and MHALT3.RV having the corresponding nucleic acid sequences as follows:

5′GGGGATATCCACCATGG(AG)ATG(CG)AGCTG(TG)GT(CA)AT(CG)CTCTT

5 ′GGGGATATCCACCATG(AG)ACTTCGGG(TC)TGAGCT(TG)GGTTTT

5 ′GGGTATATCCACCATGGCTGTCTTGGGGCTGCTCTTCT

The V_{L} domain-encoding cDNA was obtained by using the commercially available Pharmacia Mouse ScFv Module/Recombinant Phage Antibody System. A V_{L} cDNA was amplified by PCR using Taq DNA polymerase (Promega), the C region MCₖ AS.XBA antisense primer having the following sequence

5′GCGTCTAGAACTGGATGGTGGGAAGATGGA

and five universal sense primers complementary to the N-terminus of V_{L} leader sequences MLALT1.RV, MLALT2.RV, MLALT3.RV, MLALT4.RV, MLALT.5 having the following sequences.

5′GGGGATATCCACCATGGAGACAGACACACTCCTGCTAT

5′GGGGATATCCACCATGGATTTTCAAGTGCAGATTTTCAG

5′GGGGATATCCACCATGGAG(TA)CACA(GT)(TA)CTCAGGTCTTT(GA)TA

5′GGGGATATCCACCATG(GT)CCCC(AT)(GA)CTCAG(CT)T(CT)CT(TG)GT

5′GGGGATATCCACCATGAAGTTGCCTGTTAGGCTGTTG

PCR amplification of both V_{H} and V_{L} cDNAs was carried out in a MicroCycler thermal controller (Eppendorf) under the following conditions: 1 min denaturing (94° C.), 2 min annealing (55° C.), 2 min extension (72° C.) (for 30 cycles) followed by a final extension step of 7 min (72° C.).

The products were analyzed initially by electrophoresis in a 1.5% TAE agarose gel stained with ethidium bromide. The amplified cDNAs were then electrophoresed in a 2% low melting agarose gel in 0.5x TAE running buffer, and eluted according to the method of the Magic PCR Preps DNA Purification Kit (Promega).

Each variable domain encoding cDNA was ligated into a pT7Blue T-vector (Novagen) using T4-DNA ligase (Promega). The ligation mixture was used to transform NovaBlue competent cells (Novagen) according to protocols supplied by Novagen. Plasmid miniprep DNA was isolated using a Wizard plus Minipreps DNA purification system (Promega) according to the manufacturer's instructions. DNA samples were digested with the appropriate restriction enzymes, and analyzed in a 1.5% agarose gel electrophoresis. Six clones, containing inserts of the expected size, were sequenced in both directions using a Sequenase 2.0 sequencing kit (U.S. Biochemicals). For each cDNA, at least two identical clones were isolated.

The nucleotide and deduced amino acid sequences for the V_{L} (top) and V_{H} (bottom) domains of mAb 26-2F are shown in FIG. 1. According to the classification of Kabat et al., the V_{H} and V_{L} DNA sequences encode V_{H}IIID and VₖIII V regions respectively. Each sequence includes three complementarity determining regions and four framework regions. Underlined amino acids in FIG. 1 comprise the three complementarity determining regions. For example, the complementarity determining regions of the light chain variable region are

Arg-Ala-Ser-Glu-Ser-Val-Asp-Asn-Tyr-Gly-Ile-Ser-Phe-Met-Ser;

Ala-Ala-Ser-Asn-Gln-Gly-Ser; and

Gln-Gln-Ser-Lys-Glu-Val-Pro-Leu-Thr

with the remaining amino acid regions corresponding to the framework regions.

The complementarity determining regions of the heavy chain variable region are

Ser-Tyr-Thr-Met-Ser;

Thr-Ile-Ser-Ser-Gly-Gly-Gly-Asn-Thr-Tyr-Tyr-Pro-Asp-Ser-Val-Lys-Gly; and

Leu-Gly-Asp-Tyr-Gly-Tyr-Ala-Tyr-Thr-Met-Asp-Tyr

with the remaining amino acid regions corresponding to the framework regions. The deduced amino acid sequence of the first 16 N-terminal amino acids of each V domain is identical to that obtained by Edman degradation of the protein (data not shown). Portions of the leader sequence are not necessarily correct since they correspond to the PCR primers.

It is to be understood that the antibodies of the present invention include those having the CDRs depicted in FIG. 1, or antigenic portions thereof and further include and conservative substitutions thereof. It is well known in the art that certain amino acids in a protein may be replaced with other amino acids without significantly effecting the activity of the protein. Those substitutions are known as conservative substitutions and are included within the scope of the specific CDRs identified for the light and heavy variable chains. In addition, embodiments of the present invention include antibodies having the specific light and heavy variable chains identified in FIG. 1, and conservative substitutions thereof Embodiments of the present invention further include antibodies having segments of the proteins identified in FIG. 1 including the amino acid segments from amino acid 24 to amino acid 97 of V_{L} or the amino acid segments from amino acid 31 to amino acid 102 of V_{H}.

### EXAMPLE V

### Construction of Chimeric Genes

Chimeric antibodies that are immunologically reactive to angiogenin identified as cAb 26-2F were produced by expressing a chimeric variable region gene in combination with human constant region genes in transfectoma 26-2F (ATCC Designation CRL 12517). The light and heavy chain expression vectors pAG4622 and pAH4604 were utilized to prepare the chimeric antibodies of the present invention. The pAG4622 vector contained the genomic sequence encoding the constant region domain of the human ₖL chain and the gpt selectable marker as described in Mulligan, R. C. & Berg, P. (1981) Proc. Natl. Acad. Sci. USA 78, 2072-2076. The pAH4604 vector contained the hisD selectable marker as described in Hartman, S. C. & Mulligan, R. C. (1988) Proc. Natl. Acad. Sci. USA 85, 8047-8051 in addition to sequences encoding the human H chain γ1 constant region domain. The promoter region in each vector was derived from the antidansyl mAb 27-44 as described in Coloma, M. J., Hastings, A., Wims, L. A. & Morrison, S. L. (1992)J. Immunol. Methods 152, 89-104.

V_{H} and V_{L} cDNAs were modified at their 3′ end by removing the N-terminal sequence of the murine constant region and adding a splicing signal sequence at the V_{L}3′ end. For each of the modified V_{H} and V_{L} domains, cDNA from two identical independent clones was excised with either EcoRV and Sal I (for V_{L}) or EcoRV and Nhe I (for V_{H}), and gel purified.

The V_{L} and V_{H} cDNA products were ligated into pAG4622 and pAH4⁶⁰4, respectively. Several clones, isolated from HB101 competent cell (Promega) transformation, were analyzed with appropriate restriction enzymes. Recombinant vectors were isolated in duplicate from two distinct clones, each of which derived from independent V_{L}- or V_{H}-containing plasmid clones. Prior to transfection, the recombinant vectors were linearized with Pvu I isoschizomer BspCI restriction enzyme (Stratagene) and gel purified.

The V_{H} cDNA was amplified by PCR using the H chain sense primer MHALT2.RV and the H chain antisense primer H-P2. MHALT2.RV hybridized to the N-terminus of the H chain leader sequence and contains an Eco RV restriction site to facilitate cloning of the resulting PCR product into the H chain expression vector. H-P2 (CTAGCTAGCTGAGGAGACGGTGACTGAGGTTCCT) hybridized to the J region and contained a Nhe I site to allow the PCR product to be conveniently cloned into the C_{H}I region of the H chain expression vector.

The V_{L} cDNA was amplified by PCR using the L chain sense primer L-P2-sense and the L chain antisense primer L-P2-antisense. L-P2-sense (GGGGATATCCACCATGGAGACAGACACACTCCTGCRATGGGTCCTGCT) corresponds to oligonucleotide MLALT 1.RV and contains a 10 nucleotide extension at the 3′ end that hybridizes to the N-terminus of the L chain leader sequence. An EcoRV site is present in L-P2 to facilitate cloning of the PCR product into the L chain expression vector. L-P 2-antisense having the sequence AGCCGTCGACTTACGTTTCAGCTCCAGCTTGGTCCCAG hybridizes to the J region, and contains both a splicing signal sequence and a Sal I site for cloning into the intronic sequence of the L chain expression vector.

The gel-purified PCR products were cloned using the pT7Blue T-vector and independent clones were sequenced. Sequence analysis confirmed that the expected DNA assembly had been achieved, and that mutations had not been introduced during the production of these chimeric genes.

### EXAMPLE VI

### Production, Isolation and Analysis of Chimeric Antibodies

The chimeric heavy and light chain expression plasmids were cotransfected into SP2/0 or P3X non-producing myeloma cells by electroporation as described in Coloma, M. J., Hastings, A., Wims, L. A. & Morrison, S. L. (1992) J. Immunol. Methods 152, 89-104. The murine nonproducing myeloma cell lines P3×63-Ag8.653 (P3X) (CRL 1580) and Sp2/0 (CRL 1581) were obtained from the American Type Culture Collection. All cells were maintained in Dulbecco's modified Eagle's medium supplemented with 2 mM L-glutamine, 10% heat-inactivated fetal bovine serum, and antibiotics (growth medium).

Following transfection the cells were incubated on ice for 10 min, diluted in growth medium, and placed into 96-well tissue culture plates (1×10⁴ cells/well). The cells were refed 48 hr later with growth medium containing histidinol (Sigma) at a final concentration of 5 and 10 mM for SP2/0 and P3X cells, respectively. Histidinol was used to select for the presence of the hisD marker. After approximately 14 days, supernatants from growing colonies were screened by ELISA for the presence of chimeric antibodies. Two chimeric antibody producing master wells containing transfectants designated P4-5 and S13-1, were obtained from transfected P3X or SP2/0 cells, respectively. P4-5 and S13-1 were subcloned twice by limiting dilution.

To obtain sufficient material for further analysis, mice were primed with a 1 ml intraperitoneal injection of pristane followed 7 days later with an intraperitoneal injection of either P4-5 and S13-1 cells (1×10⁶), and the cAb 26-2F obtained from each of the transfectoma cell types (ATCC Designation CRL 12517) was purified from ascites fluid by affinity chromatography using GammaBind Plus Sepharose (Pharmacia). Ascites fluid (80 ml) was diluted 1:1 with PBS, and clarified by centrifugation. The supernatant was filtered through a glass fiber filter and a 0.2 μm cellulose nitrate filter. After a further dilution with PBS to a final volume of 400 ml, the antibodies were adsorbed onto the gel, washed with PBS, and eluted with 0. 1 M glycine-HCl into tubes containing an appropriate amount of 1 M Tris-HCl to neutralize the supernatant. Following dialysis against 0.9% NaCl, the antibodies were quantified by enzyme-linked immunoadsorbent assay (ELISA, described below), and stored at −70° C. MOPC 31C, a non-specific IgGIκ-secreting mouse hybridoma (CCL 130, American Type Culture Collection) was propagated, and IgG purified from ascites.

Chimeric antibody producing transfectomas were detected by a modification of the screening ELISA protocol described in Fett et al. previously cited. Briefly, the wells of a 96-well plate were coated with affinity purified goat anti-human IgG Fc (γ-chain specific) goat anti-human κ chain (each at 10 μg/ml, Organon Teknika), or human angiogenin (1 μg/ml). Following a blocking step in which the wells were incubated with a solution of 0.5% ovalbumin, 50 μl of culture supernatant (diluted 1:1 with 0.25% ovalbumin) was added. Following a 2 hr incubation period at room temperature, plates were washed with PBS containing 0.5% Tween 20, and alkaline phosphatase-labeled goat anti-human IgG (Kirkegaard and Perry) was added to each well. Plates were incubated at room temperature for 1 hr. and washed as above. p-Nitrophenyl phosphate (1 mg/ml, 100 μl/well) in diethanolamine buffer at pH 9.8 was then added and the reaction was stopped 1 hr. later by the addition of 3 N NaOH. The absorbance was measured on a Dynatech MR600 ELISA plate reader at 405 nm, with a turbidity reference of 630 nm.

A modified protocol for competition radioimmunoassay (RIA) for binding affinity was used to determine the IC₅₀ of the antibodies. The term “IC₅₀” refers to the concentration of unlabeled angiogenin at which the binding of an iodinated derivative of angiogenin is decreased by 50%. The RIA was performed according to the following method. To measure the binding affinity of mAb 26-2F, plates were coated with 50 μl of goat anti-mouse IgG Fc (γ-chain specific, Organon Teknika) per well. To measure the binding affinity of the chimeric antibody, plates were coated with 50 μl of goat antihuman IgG Fc (see above) per well. Both antibodies were prepared as 10 μg/ml solutions in borate coating buffer. Radioactivity was determined using a Micromedic 4/600 plus Gamma Counter. S 13-1 and P4-5-derived cAb 26-2F have IC₅₀ values of 2.1×10-9 M and 2.4×10 ⁻⁹ M, respectively. These values are essentially indistinguishable from that of mAb 26-2F (1.6×10⁻⁹ M) within the error of the assay.

The general procedures for SDS-polyacrylamide (10%) gel electrophoresis, transfer, and Western blotting have been described in Kurachi, K. Rybak, S. M., Fett, J. W., Shapiro, R., Strydom, D. J., Olson, K. A., Riordan, J. F., Davie, E. W. & Vallee, B. L. (1988) Biochemistry 27, 6557,6562. Samples were boiled in a buffer containing 5% P-mercaptoethanol prior to loading onto the gel. For detection of human components, goat anti-human IgG Fc and κ chain antibodies were used. Western blot analysis under reducing or non-reducing conditions using reagents specific for human κ and γ1C region determinants demonstrated that cAb 26-2F from either transfectoma cell source contained chimeric light and heavy chains of the expected molecular weights.

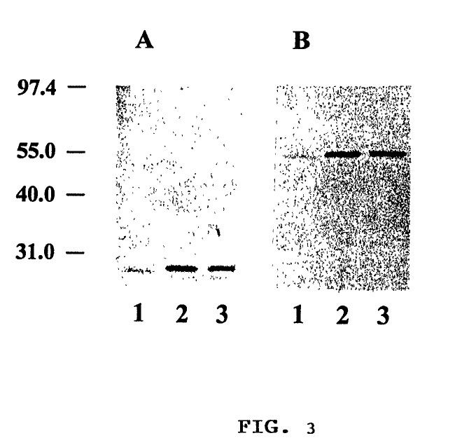

Reduced proteins (400 ng) were separated by SDS-polyacrylamide gel electrophoresis (10%) and transferred to nitrocellulose membranes. The nitrocellulose membrane was incubated with either goat anti-human κ chain (A) or goat anti-human IgG Fc-specific (B) antibodies followed by treatment with alkaline phosphatase-labeled rabbit anti-goat IgG and nitroblue tetrazolium. As shown in FIG. 3, the gel loading order is as follows: Lane 1, mAb 26-2F; lane 2, cAb 26-2F from S13-1; lane 3, cAb 26-2F from P4-5. The migration pattern of molecular weight standards (X 10⁻³) is indicated on the left.

Immunoglobulin chains were visualized with alkaline phosphatase-labeled rabbit anti-goat IgG with nitroblue tetrazolium as a substrate. Immunoglobulin concentrations were determined spectroscopically assuming that a 1 mg/ml solution has an absorbance of 1.43 at 280 nm.

Under reducing conditions chimeric light chains of the expected molecular weight (approximately 25,000 and 55,000 daltons) were observed when cAb 26-2F derived from either S13-1 or P4-5 was analyzed. Under nonreducing conditions cAb 26-2F derived from either S13-1 or P4-5 migrated to a position corresponding to 160,000 daltons (data not shown) thus indicating that the chimeric L and H chains were correctly assembled into complete H₂L₂ molecules.

S13-1 or P4-5 transfectoma cells were injected into pristane-primed athymic mice to generate ascites fluid. Antibody was then subsequently isolated by protein G-Sepharose affinity chromatography. The total yield of purified cAb 26-2F from either transfectoma source was approximately 3 mg/mouse.

Purified S13-1 and P4-5-derived chimeric antibodies were subjected to ten cycles of Edman sequence analysis. Light and heavy chain N-terminal amino acids of both chimeric antibodies were determined to be identical (data not shown) and to correspond to the N-terminal amino acids of mAb 26-2F.

### EXAMPLE VII

### Angiogenesis Inhibition

The following assays were performed to determine the ability of chimerized antibodies to inhibit angiogenin functions.

The chick chorioallantoic membrane (CAM) assay was used according to Fett et al. previously cited. A comparison of the ability of cAb 26-2F and its murine counterpart to inhibit the angiogenic activity of angiogenin on the CAM is shown in Table 1 below. The combined data represents 3 sets of assays. Each individual assay employed between 15 and 19 eggs. The amount applied per egg is 10 ng of angiogenin and 100 ng of IgGs. The assay results are expressed as the ratio of positive to total surviving eggs with the percentage of positive eggs given in parentheses. The significance p was calculated from χ² values of data recorded at 48±2hr based on comparison with water controls tested simultaneously (10 positive eggs/46 total surviving eggs, 22% positive). To be designated, active samples must have a value of p<0.05.

TABLE 1

| | | mAb | | | MOPC | Re- | | |
|---|---|---|---|---|---|---|---|---|
| Group | Ang | 26-2F | S13-1 | P4-5 | 31C | sults | p | Status |
| I | + | − | − | − | − | 25/45 | 0.0009 | active |
| | | | | | | (56) | | |
| II | + | + | − | − | − | 10/45 | 0.9556 | inactive |
| | | | | | | (22) | | |
| III | + | − | + | − | − | 11/46 | 0.8038 | inactive |
| | | | | | | (24) | | |
| IV | + | − | − | + | − | 11/45 | 0.7594 | inactive |
| | | | | | | (24) | | |
| V | + | − | − | − | + | 26/45 | 0.0004 | active |
| | | | | | | (58) | | |
| VI | − | + | − | − | − | 9/42 | 0.9718 | inactive |
| | | | | | | (21) | | |
| VII | − | − | + | − | − | 7/45 | 0.4492 | inactive |
| | | | | | | (16) | | |
| VIII | − | − | − | + | − | 13/42 | 0.3258 | inactive |
| | | | | | | (31) | | |
| IX | − | − | − | − | + | 15/45 | 0.2154 | inactive |
| | | | | | | (33) | | |

Statistical analysis by the x² test indicates that cAb 26-2F purified from either S13-1 (group III, p=0.8038) or P4-5 (group IV, p=0.7594) is as potent as mAb 26-2F (group II, p=0.9556) in inhibiting the biologic activity of an equimolar amount of angiogenin. The control MOPC 31C is not inhibitory (group V, p=0.0004). Angiogenin alone is highly active (group I, p=0.0009), while the immunoglobulins alone are inactive on the CAM (groups VI-IX, p's>0.05).

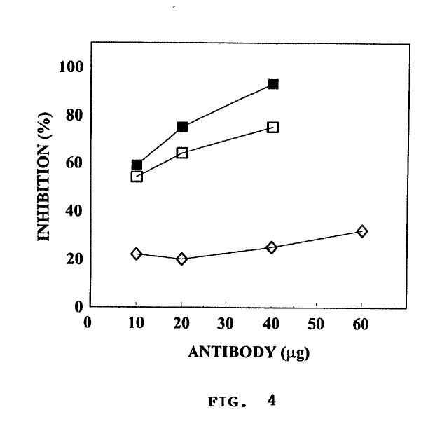

Inhibition of the ribonucleolytic activity of angiogenin by mAb 26-2F(▪), cAb 26-2F (□), or control MOPC 31 C (⋄) is shown in FIG. 4. The capacity of cAb 26-2F to inhibit tRNA degradation by angiogenin was determined by measuring the rate of formation of perchloric add-soluble fragments as described in Shapiro, R., Weremowicz, S., Riordan, J. F. & Vallee, B. L. (1987) Proc. Natl. Acad. Sci. USA 84, 8783-8787. Angiogenin was preincubated with the indicated amounts of immunoglobulins and assays were performed in 33 mM Hepes/33 mM NaCl, pH 6.8, at 37° C. At 10 μg, the two angiogenin-specific antibodies are equally inhibitory while at higher concentrations cAb 26-2F is only slightly less active.

### EXAMPLE VIII

### In Vitro Antitumor Activity

Direct cytotoxicity of cAb 26-2F toward MDA-MB-435 and MCF-7 cells was examined using a [³H] thymidine assay as described in Olson, K. A., French, T. C., Vallee, B. L. & Fett, J. W. (1994) Cancer Res. 54, 4576-4579. No evidence of direct cytotoxicity (as reflected in a decrease in [³H]thymidine uptake) of either cell type following a 48 hr incubation with cAb 26-2F (150 μg/ml) was observed.

### EXAMPLE IX

### In Vivo Antitumor Activity

Antitumor activity in vivo was assessed by using a modified version of the orthotopic model of human breast cancer tumor growth wherein tumor growth in athymic mice is measured as described by Price, J. E., Polyzos, A., Zhang, R. D. & Daniels, L. M. (1990) Cancer Res. 50, 717-721.

The estrogen-sensitive MCF-7 and estrogen-insensitive MDA-MB-435 human breast cancer cell lines were supplied by Drs. Marc E. Lippman (Georgetown University Medical Center) and Isaiah J. Fidler (University of Texas M.D. Anderson Cancer Center), respectively. It has been determined that both cell lines secrete angiogenin in vitro. All cells were maintained in Dulbecco's modified Eagle's medium supplemented with 2 MM L-glutamine, 10% heat-inactivated fetal bovine serum, and antibiotics (growth medium).

Female athymic mice were obtained at 5 weeks of age from the isolator bred colony of Charles River Laboratories at Wilmington, Mass., and maintained under specific pathogen-free conditions in a temperature- and humidity controlled environment. Experiments were begun one week later.

Tumor cells (MDA-MB-435 or MCF-7) were harvested by standard trypsinization procedures, washed in Hanks' buffered salt solution, and counted by trypan blue exclusion hemacytometry. Viable cells (MDA-MB-435, 5×10⁵ in 10 μl or MCF-7, 1×10⁶ in 20 μl ) were injected into the surgically exposed mammary fat pad using a manual repeating dispenser (Hamilton). For MCF-7 cells a pellet of 17β-estradiol (0.72 mg/pellet, 60-day release; Innovative Research of America) was placed 1 cm from the site of tumor cell injection as the source of standard estrogen supplementation. The incision was closed with an autoclip and local subcutaneous treatment was begun within 30 min. Tumor growth was monitored by caliper measurements.

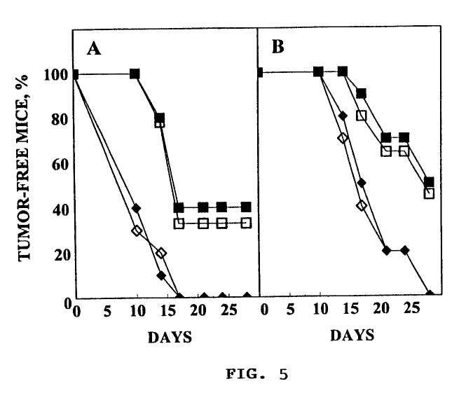

FIG. 5 demonstrates the ability of mAb 26-2F or cAb 26-2F to prevent tumor formation in mice injected with either MDA-MB-435 (A) or MCF-7 (B). Tumor cells [5×10⁵ (A) or 1×10⁶ /mouse (B)] were injected into the surgically exposed mammary fat pad on day 0. For MCF-7 cells, a 17β-estradiol pellet was implanted in each mouse as a source of exogenous estrogen. Within 30 min of tumor cell injection the mice were treated with local subcutaneous injections of either PBS (♦) or immunoglobulins [mAb 26-2F (▪), cAb 26-2F (□), MOPC 3 IC (⋄); 240 μg/dose (A) and (B)]. Mice were then treated locally with 120 μg/dose (A) or 240 μg/dose (B) 6 times per week until sacrifice on day 28. n=10 for all groups.

The results depicted in FIG. 5 indicate that cAb 26-2F is as effective as mAb 26-2F in preventing the formation of tumors of human breast cancer origin. Whereas all PBS-treated and control MOPC 31C-treated mice develop MDA-MB-435 (FIG. 5A) or MCF-7 (FIG. 5B) tumors by days 17 and 28, respectively, the chimeric and murine antibodies completely prevent the appearance of tumors in approximately 40% (MDA-MB-435) and approximately 50% (MCF-7) of the treated mice.

### EXAMPLE X

### Construction of Humanized Genes

According to an alternate embodiment of the present invention, humanized antibodies to angiogenin may be constructed by introducing nonhuman complementarity determining regions (CDRs), such as those derived from the murine mAb 26-2F into a human variable framework region with or without combining the resulting antibody with a human constant region. Such humanization procedures are known in the art. See U.S. Pat. No. 5,693,762 hereby incorporated by reference in its entirety. See also, Reichmann, Clark, Waldmann and Winter, Nature, vol. 332, pp. 323-327 (1988); Co and Queen, Nature, vol. 351, p. 501-502 (1991); Presta, Chen, O'Connor, Chisholm, Meng, Krummen, Winkler and Ferrara, Cancer Research 57, 4593-4599 (1997) and Winter and Milstein, Nature, vol. 349, pp. 293-299 (1992) each hereby incorporated by reference in their entireties.

Human variable regions that are homologous in sequence to the murine variable regions of mAb 26-2F are chosen from a data base in order to provide the human framework which will be less likely to distort the complementarity determining regions derived from the murine mAb 26-2F. Computer and molecular modelling of the antibody is used to identify the few mouse amino acid residues that make the key contacts with the CDRs. Those amino acids that make key contacts with the CDRs or otherwise contribute to the integrity or stability of the CDR regions are introduced into the human framework along with the CDRs themselves. A complex consisting of a Fab fragment of mAb 26-2F and human angiogenin is then crystallized and the critical antigen contacting amino acids in the mouse antibody are identified. At least those critical murine amino acids are incorporated into the human framework.

To produce a humanized antibody that is immunologically reactive with angiogenin according to the invention, the variable regions of mAb 26-2F are cloned, sequenced and subcloned into expression vectors according to the methods previously described for the preparation of the chimeric antibody cAb 26-2F.

The hypervariable regions of the cAb 26-2F were identified according to the method and classification of Kabat et al. (27). The V_{H} and V_{L} DNA sequences encode V_{H}IIID and V_{K} IIIV regions, respectively.

A crystallized complex of Fab fragments of mAb 26-2F bound to human angiogenin was created and analyzed. Fab fragments from mAb 26-2F were generated by using the commercially-available “ImmunoPure Fab Preparation kit” (Pierce, Rockford, Ill.). Briefly, native mAb 26-2F was digested overnight at 37° C. with the proteolytic enzyme papain. The mixture of digestion products was diluted in a binding buffer, and applied to a column containing immobilized Protein A. Material that passed through the column and did not bind to the Protein A was designated as the “Fab fragment” of the intact IgG. This solution was concentrated using a Millipore Ultrafree Biomax concentration device.

The Fab fragment of mAb 26-2F was used in crystallization studies according to the hanging-drop method described in Acharya, K. R., Subramanian, V., Shapiro, R., Riordan, J. F., and Vallee, B. L. (1992), J. Mol. Biol. 228, 1269-1270 hereby incorporated by reference in its entirety.

Working at the level of the gene and using three large mutagenic oligonucleotides for each variable domain, the mouse hypervariable regions are mounted in a single step on the human heavy-or light-chain framework regions. The reshaped human heavy-and light-chain variable domains are assembled with constant domains in three stages. This permits a step-wise check on the reshaping of the heavy-chain variable domain (stage 1), the selection of the human isotype (stage 2), and the reshaping of the light-chain variable domain and the assembly of human antibody (stage 3).

The plasmid constructions are genomic, with the sequences encoding variable domains cloned as HindIII-Bam HI fragments and those encoding the constant domains as BamHI-Bam HI fragments in either pSVgpt (heavy chain) or pSVneo (light chain) vectors. The heavy-chain enhancer sequence is included on the 5′ side of the variable domain, and expression of both light and heavy chains is driven from the heavy-chain promoter and the heavy chain signal sequence.

The humanized antibody genes are introduced into cells and expressed, purified and characterized as previously described for the chimeric antibodies of the present invention.

It is to be understood that the embodiments of the present invention which have been described are merely illustrative of some of the applications of the principles of the invention. Numerous modifications may be made by those skilled in the art based upon the teachings presented herein without departing from the true spirit and scope of the invention.

What is claimed is:

1. An antibody immunologically reactive to angiogenin or a fragment of angiogenin comprising light and heavy chain nonhuman-derived complementarity determining regions having a binding affinity to the angiogenin or fragment of angiogenin in combination with human derived polypeptide regions.

2. The antibody of claim 1 wherein the light chain complementarity determining regions have at least 80% homology to members selected from the group consisting of:
Arg-Ala-Ser-Glu-Ser-Val-Asp-Asn-Tyr-Gly-Ile-Ser-Phe-Met-Ser;
Ala-Ala-Ser-Asn-Gln-Gly-Ser; and
Gln-Gln-Ser-Lys-Glu-Val-Pro-Leu-Thr.

3. The antibody of claim 1 wherein the heavy chain complementarity determining regions have at least 80% homology to members selected from the group consisting of:
Ser-Tyr-Thr-Met-Ser;
Thr-Ile-Ser-Ser-Gly-Gly-Gly-Asn-Thr-Tyr-Tyr-Pro-Asp-Ser-Val-Lys-Gly; and
Leu-Gly-Asp-Tyr-Gly-Tyr-Ala-Tyr-Thr-Met-Asp-Tyr.

4. The antibody of claim 1 further comprising non-human derived variable light and heavy chain regions.

5. The antibody of claim 4 wherein the light chain variable region comprises an amino acid sequence having at least 80% homology to:
Met-Glu-Thr-Asp-Thr-Leu-Leu-Leu-Trp-Val-Leu-Leu-Leu-Trp-Val-Pro-Gly-Ser-Thr-Gly-Asp-Ile-Val-Leu-Thr-Gln-Ser-Pro-Ala-Ser-Leu-Ala-Val-Ser-Leu-Gly-Gln-Arg-Ala-Thr-Ile-Ser-Cys-Arg-Ala-Ser-Glu-Ser-Val-Asp-Asn-Tyr-Gly-Ile-Ser-Phe-Met-Ser-Trp-Phe-Gln-Gln-Lys-Pro-Gly-Gln-Pro-Pro-Lys-Leu-Leu-Ile-Tyr-Ala-Ala-Ser-Asn-Gln-Gly-SerGly-Val-Pro-Ala-Arg-Phe-Ser-Gly-Ser-Gly-Ser-Gly-Thr-Asp-Phe-Ser-Leu-Asn-Ile-His-Pro-Met-Glu-Glu-Asp-Asp-Thr-Ala-Met-Tyr-Phe-Cys-Gln-Gln-Ser-Lys-Glu-Val-Pro-Leu-Thr-Phe-Gly-Ala-Gly-Thr-Lys-Leu-Glu-Leu-Lys.

6. The antibody of claim 4 wherein the heavy chain variable region comprises an amino acid sequence having at least 80% homology to:
Met-Asp-Phe-Gly-Leu-Ser-Trp-Val-Phe-Leu-Val-Leu-Ile-Leu-Lys-Gly-Val-Gln-Cys-Glu-Val-Met-Leu-Cal-Glu-Ser-Gly-Gly-Gly-Leu-Val-Lys-Pro-Gly-Gly-Ser-Leu-Lys-Leu-Ser-Cys-Ala-Ala-Ser-Gly-Phe-Ser-Ser-Tyr-Thr-Met-Ser-Trp-Val-Arg-Gln-Thr-Pro-Glu-Lys-Arg-Leu-Glu-Trp-Val-Ala-Thr-Ile-Ser-Ser-Gly-Gly-Gly-Asn-Thr-Tyr-Tyr-Pro-Asp-Ser-Val-Lys-Gly-Arg-Phe-Thr-Ile-Ser-Arg-Asp-Ile-Ala-Lys-Asn-Thr-Leu-Tyr-Leu-Gln-Met-Ser-Ser-Leu-Arg-Ser-Glu-Asp-Thr-Ala-Leu-Tyr-Tyr-Cys-Thr-Leu-Gly-Asp-Tyr-Gly-Tyr-Ala-Tyr-Thr-Met-Asp-Typ-Trp-Gly-Gln-Gly-Thr-Ser-Val-Thr-Val-Ser-Ser.

7. The antibody of claim 1 wherein residues of the complementarity determining regions interact with residues 37-41 and 85-89 of angiogenin.

8. The antibody of claim 1 wherein the light chain nonhuman-derived complementarity determining region includes Asn at position 27d.

9. The antibody of claim 1 wherein the light chain nonhuman-derived complementarity determining region includes Tyr at position 28.

10. The antibody of claim 1 wherein the heavy chain nonhuman-derived complementarity determining region includes Tyr at position 100b.

11. The antibody of claim 1 wherein the heavy chain nonhuman-derived complementarity determining region includes Tyr at position 59.

12. A pharmaceutical composition comprising an antibody of claim 1 in a pharmaceutical carrier in an amount effective to inhibit the angiogenic activity of angiogenin.

13. A method for inhibiting the angiogenic activity of angiogenin comprising administering an antibody of claim 1 or a Fab or F(ab′)₂ fragment thereof, in an amount sufficient to inhibit the angiogenic activity of angiogenin.

14. An expression vector comprising a DNA encoding the antibody of claim 1.

15. A host cell transformed with the expression vector of claim 14.
